(19) United States
(12) Patent Application Publication
(10) Pub. No.: US 2009/0017557 A1
(43) Pub. Date: Jan. 15, 2009

(54) FVII Specific Antibodies and Use Thereof

(75) Inventors: Hans Kurt Pingel, Farum (DK); Else Marie Nicolaisen, Frederiksberg (DK); Janus Krarup, Gentofte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/064,843

(22) PCT Filed: Aug. 31, 2006
(86) PCT No.: PCT/EP2006/065864

§ 371 Date: Aug. 20, 2008

(30) Foreign Application Priority Data
Aug. 31, 2005 (EP) 05107963.0

(51) Int. Cl. G01N 33/566; C07K 16/18; C12N 15/11; C12N 15/00; C12N 5/06

(52) U.S. Cl. 436/501; 530/388.1; 536/23.53; 435/320.1; 435/325

(57) ABSTRACT

The present invention relates to novel antibodies against FVII, use for determining amount of correctly folded and intact FVII in a sample, as well as for purification and process optimization.

### FIELD OF THE INVENTION

The present invention relates to novel antibodies against FVII, use for determining amount of correctly folded and intact FVII in a sample, as well as for purification and process optimization.

### BACKGROUND OF THE INVENTION

For the industrial production of proteins it is desirable to be able to determine the concentration of a FVII polypeptide in a sample in a convenient and easy assay. One way to do this is by specific antibodies that will bind to the FVII polypeptide and which can subsequently be quantified by enzymatic reaction of a conjugated enzyme. This enzyme linked immunosorbent assay (ELISA) is well known in the art for detection of specific proteins in a sample. For a more precise antigen concentration determination where the absolute amounts of an antigen in a sample is to be determined and an antigen standard is available the “sandwich” ELISA is very useful.

To utilize this assay, one antibody (the “capture” antibody) is purified and bound to a solid phase. Antigen is then added and allowed to complex with the bound antibody. Unbound products are then removed with a wash, and labeled second antibody (the “detection” antibody) is allowed to bind to the antigen, thus completing the “sandwich”. The assay is then quantified by measuring the amount of labeled second antibody bound to the matrix, through the use of a calorimetric substrate. A major advantage of this technique is that the antigen does not need to be purified prior to use, and also that these assays are very specific. However, not all antibodies can be used. Monoclonal antibody combinations must be qualified at “matched pairs”, meaning that they can recognize separate epitopes on the antigen.

The sensitivity of the sandwich ELISA is dependent on four factors: The number of molecules of the first antibody that are bound to the solid phase; the affinity of the first antibody for the antigen; the affinity of the second antibody for the antigen; the specific activity of the second antibody.

Especially the affinity of the antibodies for the antigen can only be altered by substitution with other antibodies. Thus antibodies with strong affinity for a particular FVII polypeptide are desirable.

Many proteins require post translational modifications in order to be active. These modifications include cleavage of pro-peptides and correct folding of the mature polypeptide.

A particular family of proteins is recognized by a characteristic modular organization and requires vitamin K for their biosynthesis. The amino-terminal membrane-binding domain contains gamma-carboxylated glutamic acid (GLA) residues, post-translationally modified by a carboxylase in a vitamin K dependent reaction. Gamma-carboxylation of these proteins affects their proper folding and therefore also their activity.

During production of proteins belonging to this family, it is sometimes desirable to purify culture liquids in a very efficient way by the application of immunoaffinity columns. Also in order to optimize the yield of active FVII polypeptide in the culture, it would be desirable to better control the culturing process. This objective would be reached by identification of high efficient monoclonal antibodies and an easy and quick assay for the determination of the ratio of active protein (correctly processed: gamma-carboxylation leading to formation of structural epitopes) of interest to total amount of the FVII polypeptide in the culture. Thereby the process may be monitored and adjusted to optimal conditions such as allowing harvest of the culture at the optimal time during culture.

### SUMMARY OF THE INVENTION

Specific monoclonal antibodies having high affinity in the presence of a divalent cation such as calcium towards the GLA domain of FVII have now been identified.

These antibodies may be utilized in methods for good absolute concentration determinations of a FVII polypeptide and further when these high affinity antibodies are combined with antibodies that recognize different epitopes exposed on the FVII polypeptide a method has been developed which is capable of determining the ratio of correctly processed FVII polypeptide to total FVII polypeptide in a sample. This method may be used for optimizing the yield of active FVII polypeptide during production.

Furthermore these novel specific antibodies having high affinity in the presence of calcium towards the GLA domain may be used for very efficient and simple methods for purification.

Thus in a broad aspect the present invention relates to the identification of high efficient monoclonal antibodies against wild type human FVII.

A first aspect of the present invention relates to a monoclonal antibody that binds to an epitope present on an intact gamma-carboxyglutamic acid (GLA) domain of wild type human FVII only in the presence of at least 0.05 mM of a divalent cation.

A second aspect of the invention relates to a nucleic acid molecule encoding a monoclonal antibody that binds to an epitope present on an intact gamma-carboxyglutamic acid (GLA) domain of wild type human FVII only in the presence of at least 0.05 mM of a divalent cation.

In a further aspect the present invention relates to a vector comprising the nucleic acid molecule encoding a monoclonal antibody that binds to an epitope present on an intact gamma-carboxyglutamic acid (GLA) domain of wild type human FVII only in the presence of at least 0.05 mM of a divalent cation.

In a further aspect the present invention relates to a cell comprising a vector comprising the nucleic acid molecule encoding a monoclonal antibody that binds to an epitope present on an intact gamma-carboxyglutamic acid (GLA) domain of wild type human FVII only in the presence of at least 0.05 mM of a divalent cation.

In a further aspect the present invention relates to a method for determining the amount of FVII polypeptides comprising an intact GLA domain in a sample the method comprising the steps of:

a) bringing the sample in the presence of at least 0.05 mM of a divalent cation in contact with a first monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the first monoclonal antibody to form a first antibody complex,
c) bringing the first antibody complex in contact with a detectable second monoclonal antibody specific for a second epitope present on the FVII polypeptide, the second epitope being different from the epitope of the first monoclonal antibody,
d) allowing the first antibody complex to bind to the detectable second monoclonal antibody to form a second antibody complex, and
e) detecting the amount of the second antibody complex by detecting the amount of second monoclonal antibody present in the second antibody complex.

In a further aspect the present invention relates to a method for determining the amount of FVII polypeptides comprising an intact GLA domain in a sample the method comprising the steps of:

a) bringing the sample in contact with a second monoclonal antibody specific for an epitope present on the FVII polypeptide, the epitope being different from the epitope identified by a monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the second monoclonal antibody to form a first antibody complex,
c) bringing the first antibody complex in the presence of at least 0.05 mM of a divalent cation in contact with a detectable first monoclonal antibody that binds to an epitope present on an intact gamma-carboxyglutamic acid (GLA) domain of wild type human FVII only in the presence of at least 0.05 mM of a divalent cation,
d) allowing the first antibody complex to bind to the detectable first monoclonal antibody to form a second antibody complex, and
e) detecting the amount of the second antibody complex by detecting the amount of the first monoclonal antibody present in the second antibody complex.

In a further aspect the present invention relates to a method for determining the ratio of FVII polypeptides comprising an intact GLA domain to total amount of the FVII polypeptide in a sample comprising the steps of:

a) determining the amount of the FVII polypeptides comprising an intact GLA domain by use of method according to the invention; and
b) determining the total amount of FVII polypeptide present in the sample.

In a further aspect the present invention relates to the use of a method according to the invention, for optimizing the yield of the functional FVII polypeptide during production.

In a further aspect the present invention relates to a method for the purification of FVII polypeptides comprising an intact GLA domain from a sample the method comprising the steps of:
(a) coupling of a monoclonal antibody that binds to an epitope present on an intact gamma-carboxyglutamic acid (GLA) domain of wild type human FVII only in the presence of at least 0.05 mM of a divalent cation to an immunoaffinity purification column,
(b) applying the sample to the column in the presence of at least 0.05 mM of a divalent cation,
(c) eluting the FVII polypeptides comprising an intact GLA domain from the column by removal of the divalent cation from the column.

### DESCRIPTION OF FIGURES

The invention is explained in detail below with reference to the drawing(s), in which

FIG. 1 shows the full amino acid sequence of native human coagulation Factor VII (SEQ ID NO:1).

FIG. 2 shows the nucleotide sequences and amino acid sequences of the mature variable light (VL) and variable heavy (VH) regions of exemplary antibodies according to the invention.

FIG. 3 shows a typical standard curve for the described ELISA assay.

FIG. 4 shows typical In/In standard curve for the described ELISA assay.

FIG. 5 shows CDI as a function of time (in days) for different cultivations.

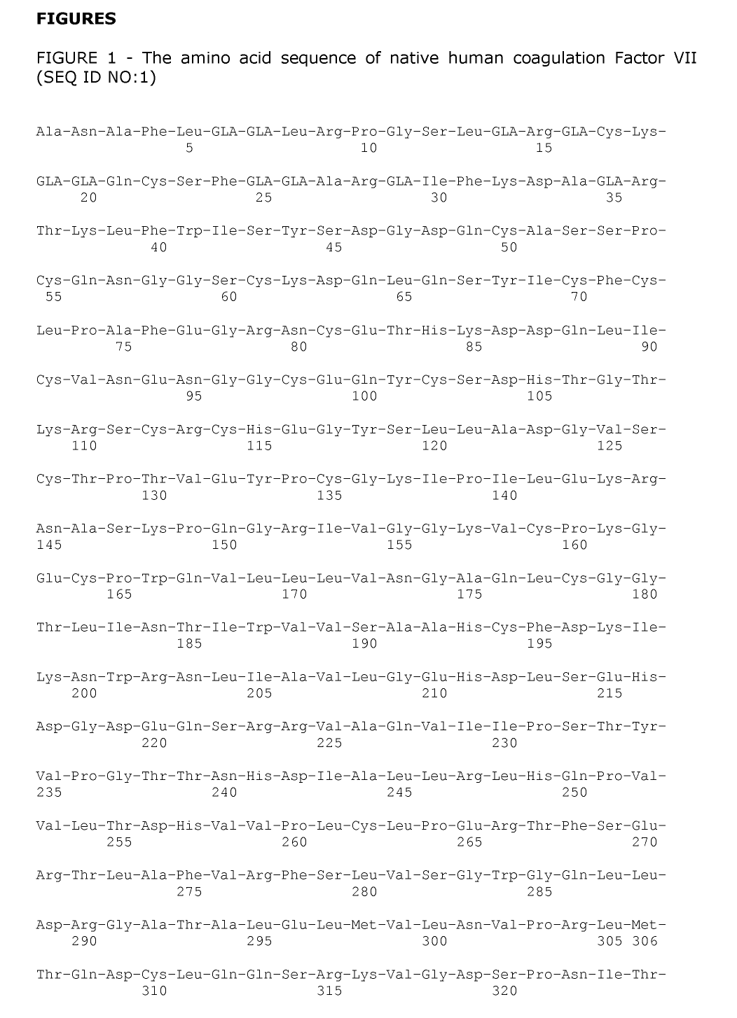

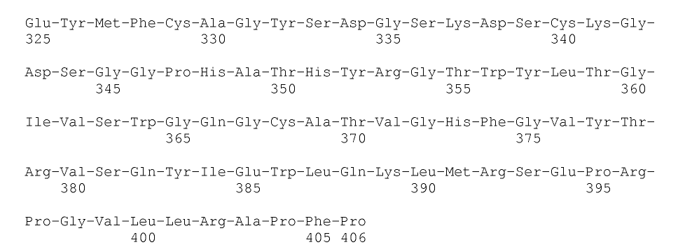

### DETAILED DESCRIPTION OF THE INVENTION

The present invention relates in a broad aspect to the identification of new specific antibodies against the GLA domain of FVII.

These specific monoclonal antibodies have a high affinity towards the GLA domain of FVII in the presence of a divalent cation such as calcium.

The antibodies may be utilized in methods for good absolute concentration determinations of a FVII polypeptide with an intact GLA domain and further when these high affinity antibodies are combined with antibodies that recognizes different epitopes exposed on the FVII polypeptide a method has been developed which is capable of determining the ratio of correctly processed FVII polypeptide to total FVII polypeptide in a sample. This method may be used for optimizing the yield of active FVII polypeptide during production.

Furthermore these novel specific antibodies having high affinity in the presence of a divalent cation such as calcium towards the GLA domain may be used for very efficient and simple methods for purification.

In one aspect of the invention, the antibodies according to the invention are used for determination of amounts of FVII polypeptides with an intact GLA domain. This is typically achieved with an assay, such as an ELISA assay, wherein a first antibody, the catching antibody is attached to a solid support followed by binding of the antibody to the FVII polypeptide under given proper conditions. Following a washing step the FVII polypeptide will be retained on the solid support. Unbound products are removed by the wash, and labeled second antibody (the “detection” antibody) is allowed to bind to the FVII polypeptide, the FVII polypeptide, thus completing the “sandwich”. The amount of the antigen is then quantified by measuring the amount of labeled second antibody bound to the matrix. In case of a detecting antibody linked to an enzyme a calorimetric assay can be performed and a change in color determined. Having a proper standard of the FVII polypeptide with known concentration the absolute amount of the FVII polypeptide present in the sample can be determined.

In one embodiment of the invention, when the detecting antibody is enzyme linked, the method of the invention relates to a sandwich ELISA method.

In the present invention the terms “catching antibody” means the first antibody of a sandwich ELISA, diluted in buffer, which is attached passively to the solid phase on incubation. Active attachment can also be used e.g. by using a biotinylated antibody which is added to a streptavidine coated solid phase.

In the present invention the term “detecting antibody” means the second Antibody of a sandwich ELISA, diluted in a buffer, which is added after the antigen. The second antibody can be conjugated as in a direct ELISA or an anti-species conjugate as in a classic sandwich ELISA. The anti-species conjugate binds to species of the serum from which the second antibody was prepared.

Human plasma FVII consists of four discrete domains: an amino terminal (N-terminal) gamma-carboxyglutamic acid (GLA) domain (amino acids 1-38), two epidermal growth factor (EGF)-like domains, and a serine protease domain. The active two-chain enzyme is generated by specific cleavage after Arg152 (Hagen et al., Proc Natl Acad Sci USA, 1986; 83:2412-2416).

The N-terminal GLA domain binds to phospholipid surfaces; the C-terminal serine protease domain confers the enzymatic activity; the two EGF-like domains are spacers between them; all four domains contribute to the interaction with tissue factor (TF).

Calcium ions bind to three domains in FVII (Banner et al., Nature 1996; 380:41-46). Without calcium ions FVII has virtually no biological activity. Seven calcium sites are located in the GLA domain, and they need to be occupied for FVII to bind to cell membranes (Person and Petersen, Eur J Biochem 1995; 234:293-300), and also for a proper interaction with TF.

Sensitivity and thus good determinations of the absolute content and concentration of FVII molecules with and without an intact GLA domain in a culture sample is among other factors dependent on good antibodies having high affinity towards their target antigen. In a particular embodiment the antibodies of the invention for use in determining the amount of a FVII molecules with an intact GLA domain are selected as antibodies having a very high affinity towards the polypeptide in the presence of a divalent cation, such as Ca²⁺.

Other high affinity antibodies against different epitopes on FVII polypeptides than the GLA domain may also be used in these methods.

The antibodies used for good determinations of the absolute content and concentration of all FVII molecules with and without an intact GLA domains should preferably recognize epitopes which are always present in the antigen irrespective of whether the antigen is properly folded or activated. In factor VIIa, epitopes found within the EGF-like domains are particularly suited for this purpose.

As used herein, the terms “Factor VII polypeptide” or “FVII polypeptide” means any protein comprising the amino acid sequence 1-406 of wild-type human Factor VIIa (i.e., a polypeptide having the amino acid sequence disclosed in U.S. Pat. No. 4,784,950), variants thereof as well as Factor VII derivatives and Factor VII conjugates. This includes FVII variants, Factor VII derivatives and Factor VII conjugates exhibiting substantially the same or improved biological activity relative to wild-type human Factor VIIa. Such variants of Factor VII may exhibit different properties relative to human Factor VII, including stability, phospholipid binding, altered specific activity, and the like.

The terms “Factor VII” or “FVII” means Factor VII polypeptides in their uncleaved (zymogen) form. Typically, Factor VII is cleaved between residues 152 and 153 to yield Factor VIIa. “Wild type human FVII” is the uncleaved zymogen form of wild type human FVIIa in its functional bioactive form.

The terms “Factor VIIa” or “FVIIa” means Factor VII polypeptides that have been proteolytically processed to yield their respective functional bioactive forms.

As used herein, “wild type human FVIIa” is a polypeptide having the amino acid sequence disclosed in U.S. Pat. No. 4,784,950 in its functional bioactive form.

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US Patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota).

The term “improved biological activity” refers to FVII polypeptides with i) substantially the same or increased proteolytic activity compared to recombinant wild type human Factor VIIa or ii) to FVII polypeptides with substantially the same or increased TF binding activity compared to recombinant wild type human Factor VIIa or iii) to FVII polypeptides with substantially the same or increased half life in blood plasma compared to recombinant wild type human Factor VIIa. The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

Non-limiting examples of Factor VII variants having substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gladomain and exhibiting an enhanced membrane binding as disclosed in WO 99/20767, US patents U.S. Pat. No. 6,017,882 and U.S. Pat. No. 6,747,003, US patent application 20030100506 (University of Minnesota) and WO 00/66753, US patent applications US 20010018414, US 2004220106, and US 200131005, US patents U.S. Pat. No. 6,762,286 and U.S. Pat. No. 6,693,075 (University of Minnesota); and FVII variants as disclosed in WO 01/58935, US patent U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.). Examples of variants of factor VII include, without limitation, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M 298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M 298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M 298Q-FVII, F374Y/V158T/M298Q/E296V-FVII, F374Y/E296V/S314E/M 298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M 298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153Ile to 223Arg.

The antibodies used for determinations of concentration of FVII molecules with an intact GLA domain recognize an epitope in the GLA domain. One preferred epitope in the GLA domain is an epitope comprising one or more of the amino acid residues Phe4, Leu5, Gla6, Gla7, Leu8, Pro10, Gly11, Gla14, Arg15, Gla16, Cys17, Gla19, Gla20, Cys22, Gla25, Gla26, Ala27, Gla29, Phe31, Lys32, Gla35 of SEQ ID NO:1.

The phrase “an intact GLA domain” as used herein is intended to mean a GLA domain has a disulphide bond corresponding to the disulphide bond between cys17 and cys22 of human FVII.

The phrase “FVII polypeptides comprising an intact GLA domain” as used herein is intended to mean a FVII polypeptides wherein an intact GLA domain covalently attached to the rest of the FVII molecule.

Within the context of this invention, the term that an antibody “binds” a determinant designates that the antibody binds the determinant with specificity and/or affinity.

“Specific binding” or “specificity” refers to the ability of an antibody or other agent to detectably bind an epitope presented on an antigen, such as a FVII polypeptide, while having relatively little detectable reactivity with other proteins or structures. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a FVII polypeptide).

An “epitope” is an area or region on an antigen to which an antigen-binding peptide (such as an antibody) specifically binds. A protein epitope may comprise amino acid residues directly involved in the binding (also called immunodominant component of the epitope) and other amino acid residues, which are not directly involved in the binding, such as amino acid residues which are effectively blocked by the specifically antigen binding peptide (in other words, the amino acid residue is within the “footprint” of the specifically antigen binding peptide). The term epitope herein includes both types of amino acids in any particular region of a FVII polypeptide that specifically binds to an anti-FVII antibody. FVII polypeptides may comprise a number of different epitopes, which may include, without limitation, (1) linear peptide antigenic determinants, (2) conformational antigenic determinants which consist of one or more non-contiguous amino acids located near each other in a mature FVII polypeptide conformation; and (3) post-translational antigenic determinants which consist, either in whole or part, of molecular structures covalently attached to a FVII polypeptide, such as carbohydrate groups.

The phrase that a first antibody binds “substantially” or “at least partially” the same epitope as a second antibody means that the epitope binding site for the first antibody comprises at least 10%, 20%, 30%, 40%, 50%, 60%, 70%, 80%, 90%, or more of the amino acid residues on the antigen that constitutes the epitope binding site of the second antibody. Also, that a first antibody binds substantially or partially the same epitope as a second antibody means that the first and second antibodies compete in binding to the antigen, as described above. Thus, the term “binds to substantially the same epitope or determinant as” the monoclonal antibody FVII-3F3A4 means that an antibody “competes” with FVII-3F3A4. Generally, an antibody that “binds to substantially the same epitope or determinant as” the monoclonal antibody of interest (e.g. FVII-3F3A4, FVII-3F20A1, FVII-3F11A3) means that the antibody “competes” with the antibody of interest for binding to one or more FVII polypeptides.

The term “linear peptide antigenic determinants” is defined as an epitope composed of amino acid residues that are contiguous on the linear sequence of amino acids (primary structure).

The term “conformational antigenic determinants” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent the on 3-dimensional structure. The term ‘conformational’ is therefore often used interchangeably with ‘structural’.

In one embodiment the present invention relates to a monoclonal antibody that binds to an epitope present on an intact gamma-carboxyglutamic acid (GLA) domain of wild type human FVII only in the presence of at least 0.05 mM of a divalent cation, wherein the epitope comprises one or more of the amino acid residues Phe4, Leu5, Gla6, Gla7, Leu8, Pro10, Gly11, Gla14, Arg15, Gla16, Cys17, Gla19, Gla20, Cys22, Gla25, Gla26, Ala27, Gla29, Phe31, Lys32, Gla35 of SEQ ID NO:1.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody competes with an antibody comprising:

(a) a light chain variable region comprising the amino acid sequence of SEQ ID NO:3, and a heavy chain variable region comprising the amino acid sequence of SEQ ID NO:5;

(b) a light chain variable region comprising the amino acid sequence of SEQ ID NO:7, and a heavy chain variable region comprising the amino acid sequence of SEQ ID NO:9; or

(c) a light chain variable region comprising the amino acid sequence of SEQ ID NO:11, and a heavy chain variable region comprising the amino acid sequence of SEQ ID NO:13.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody comprises:

(a) a light chain CDR1 variable region comprising an amino acid sequence corresponding to residues 24-34 of the amino acid sequence of SEQ ID NO:3, a light chain CDR2 variable region comprising an amino acid sequence corresponding to residues 50-56 of the amino acid sequence of SEQ ID NO:3, a light chain CDR3 variable region comprising an amino acid sequence corresponding to residues 89-95 of the amino acid sequence of SEQ ID NO:3, and a heavy chain CDR1 variable region comprising an amino acid sequence corresponding to residues 33-35 of the amino acid sequence of SEQ ID NO:5, a heavy chain CDR2 variable region comprising an amino acid sequence corresponding to residues 50-64 of the amino acid sequence of SEQ ID NO:5, a heavy chain CDR3 variable region comprising an amino acid sequence corresponding to residues 99-112 of the amino acid sequence of SEQ ID NO:5;

(b) a light chain CDR1 variable region comprising an amino acid sequence corresponding to residues 24-34 of the amino acid sequence of SEQ ID NO:7, a light chain CDR2 variable region comprising an amino acid sequence corresponding to residues 50-56 of the amino acid sequence of SEQ ID NO:7, a light chain CDR3 variable region comprising an amino acid sequence corresponding to residues 89-95 of the amino acid sequence of SEQ ID NO:7, and a heavy chain CDR1 variable region comprising an amino acid sequence corresponding to residues 33-35 of the amino acid sequence of SEQ ID NO:9, a heavy chain CDR2 variable region comprising an amino acid sequence corresponding to residues 50-64 of the amino acid sequence of SEQ ID NO:9, a heavy chain CDR3 variable region comprising an amino acid sequence corresponding to residues 99-112 of the amino acid sequence of SEQ ID NO:9; or

(c) a light chain CDR1 variable region comprising an amino acid sequence corresponding to residues 24-34 of the amino acid sequence of SEQ ID NO:11, a light chain CDR2 variable region comprising an amino acid sequence corresponding to residues 50-56 of the amino acid sequence of SEQ ID NO:11, a light chain CDR3 variable region comprising an amino acid sequence corresponding to residues 89-95 of the amino acid sequence of SEQ ID NO:11, and a heavy chain CDR1 variable region comprising an amino acid sequence corresponding to residues 33-35 of the amino acid sequence of SEQ ID NO:13, a heavy chain CDR2 variable region comprising an amino acid sequence corresponding to residues 50-64 of the amino acid sequence of SEQ ID NO:13, a heavy chain CDR3 variable region comprising an amino acid sequence corresponding to residues 99-112 of the amino acid sequence of SEQ ID NO:13.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody comprises

(a) a light chain variable region comprising an amino acid sequence at least 50% identical to SEQ ID NO:3, and a heavy chain variable region comprising an amino acid sequence at least 50% identical to SEQ ID NO:5;

(b) a light chain variable region comprising an amino acid sequence at least 50% identical to SEQ ID NO:7, and a heavy chain variable region comprising an amino acid sequence at least 50% identical to SEQ ID NO:9; or

(c) a light chain variable region comprising an amino acid sequence at least 50% identical to SEQ ID NO:11, and a heavy chain variable region comprising an amino acid sequence at least 50% identical to SEQ ID NO:13.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody comprises

(a) a light chain variable region comprising an amino acid sequence of SEQ ID NO:3, and a heavy chain variable region comprising an amino acid sequence of SEQ ID NO: 5;

(b) a light chain variable region comprising an amino acid sequence of SEQ ID NO:7, and a heavy chain variable region comprising an amino acid sequence of SEQ ID NO:9; or

(c) a light chain variable region comprising an amino acid sequence of SEQ ID NO:11, and a heavy chain variable region comprising an amino acid sequence of SEQ ID NO:13.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody is an IgG1, IgG2, IgG3, or IgG4 antibody.

In one embodiment the present invention relates to a monoclonal antibody that binds to an epitope present on an intact gamma-carboxyglutamic acid (GLA) domain of wild type human FVII only in the presence of at least 0.05 mM of a divalent cation, which monoclonal antibody is an IgG4 antibody.

In one embodiment the present invention relates to a method for determining the amount of FVII polypeptides comprising an intact GLA domain in a sample, the method comprising the steps of:

a) bringing the sample in the presence of at least 0.05 mM of a divalent cation in contact with a first monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the first monoclonal antibody to form a first antibody complex,
c) bringing the first antibody complex in contact with a detectable second monoclonal antibody specific for a second epitope present on the FVII polypeptide, the second epitope being different from the epitope of the first monoclonal antibody,
d) allowing the first antibody complex to bind to the detectable second monoclonal antibody to form a second antibody complex, and
e) detecting the amount of the second antibody complex by detecting the amount of second monoclonal antibody present in the second antibody complex, wherein the second epitope is present on the EGF-like domain 1 or EGF-like domain 2 of the FVII polypeptide.

In one embodiment the present invention relates to a method for determining the amount of FVII polypeptides comprising an intact GLA domain in a sample the method comprising the steps of:

a) bringing the sample in the presence of at least 0.05 mM of a divalent cation in contact with a first monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the first monoclonal antibody to form a first antibody complex,
c) bringing the first antibody complex in contact with a detectable second monoclonal antibody specific for a second epitope present on the FVII polypeptide, the second epitope being different from the epitope of the first monoclonal antibody,
d) allowing the first antibody complex to bind to the detectable second monoclonal antibody to form a second antibody complex, and
e) detecting the amount of the second antibody complex by detecting the amount of second monoclonal antibody present in the second antibody complex, wherein the divalent cation is Ca²⁺ present in the range from about 0.05 mM to about 50 mM, such as from about 0.1 mM to about 30 mM, such as from about 1 mM to about 20 mM, such as from about 5 mM to about 10 mM.

In one embodiment the present invention relates to a method for determining the amount of FVII polypeptides comprising an intact GLA domain in a sample the method comprising the steps of:

a) bringing the sample in the presence of at least 0.05 mM of a divalent cation in contact with a first monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the first monoclonal antibody to form a first antibody complex,
c) bringing the first antibody complex in contact with a detectable second monoclonal antibody specific for a second epitope present on the FVII polypeptide, the second epitope being different from the epitope of the first monoclonal antibody,
d) allowing the first antibody complex to bind to the detectable second monoclonal antibody to form a second antibody complex, and
e) detecting the amount of the second antibody complex by detecting the amount of second monoclonal antibody present in the second antibody complex, wherein the detection of the detectable antibody is performed by a method selected from ELISA, surface plasmon resonance, and pieso electric biosensors.

In one embodiment the present invention relates to a method for determining the amount of FVII polypeptides comprising an intact GLA domain in a sample the method comprising the steps of:

a) bringing the sample in contact with a second monoclonal antibody specific for an epitope present on the FVII polypeptide, the epitope being different from the epitope identified by a monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the second monoclonal antibody to form a first antibody complex,
c) bringing the first antibody complex in the presence of at least 0.05 mM of a divalent cation in contact with a detectable first monoclonal antibody that binds to an epitope present on an intact gamma-carboxyglutamic acid (GLA) domain of wild type human FVII only in the presence of at least 0.05 mM of a divalent cation,
d) allowing the first antibody complex to bind to the detectable first monoclonal antibody to form a second antibody complex, and
e) detecting the amount of the second antibody complex by detecting the amount of the first monoclonal antibody present in the second antibody complex, wherein the second epitope is present on the EGF-like domain 1 or EGF-like domain 2 of the FVII polypeptide.

In one embodiment the present invention relates to a method for determining the amount of FVII polypeptides comprising an intact GLA domain in a sample the method comprising the steps of:

a) bringing the sample in contact with a second monoclonal antibody specific for an epitope present on the FVII polypeptide, the epitope being different from the epitope identified by a monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the second monoclonal antibody to form a first antibody complex,
c) bringing the first antibody complex in the presence of at least 0.05 mM of a divalent cation in contact with a detectable first monoclonal antibody that binds to an epitope present on an intact gamma-carboxyglutamic acid (GLA) domain of wild type human FVII only in the presence of at least 0.05 mM of a divalent cation,
d) allowing the first antibody complex to bind to the detectable first monoclonal antibody to form a second antibody complex, and
e) detecting the amount of the second antibody complex by detecting the amount of the first monoclonal antibody present in the second antibody complex, wherein the divalent cation is Ca²⁺ present in the range from about 0.05 mM to about 50 mM, such as from about 0.1 mM to about 30 mM, such as from about 1 mM to about 20 mM, such as from about 5 mM to about 10 mM.

In one embodiment the present invention relates to a method for determining the amount of FVII polypeptides comprising an intact GLA domain in a sample the method comprising the steps of:

a) bringing the sample in contact with a second monoclonal antibody specific for an epitope present on the FVII polypeptide, the epitope being different from the epitope identified by a monoclonal antibody that binds to an epitope present on an intact gamma-carboxyglutamic acid (GLA) domain of wild type human FVII only in the presence of at least 0.05 mM of a divalent cation,
b) allowing any of the FVII polypeptides present in the sample to bind to the second monoclonal antibody to form a first antibody complex,
c) bringing the first antibody complex in the presence of at least 0.05 mM of a divalent cation in contact with a detectable first monoclonal antibody that binds to an epitope present on an intact gamma-carboxyglutamic acid (GLA) domain of wild type human FVII only in the presence of at least 0.05 mM of a divalent cation,
d) allowing the first antibody complex to bind to the detectable first monoclonal antibody to form a second antibody complex, and
e) detecting the amount of the second antibody complex by detecting the amount of the first monoclonal antibody present in the second antibody complex, wherein the detection of the detectable antibody is performed by a method selected from ELISA, surface plasmon resonance, and pieso electric biosensors.

In one embodiment the present invention relates to a method for the purification of FVII polypeptides comprising an intact GLA domain from a sample the method comprising the steps of:
(a) coupling of a monoclonal antibody that binds to an epitope present on an intact gamma-carboxyglutamic acid (GLA) domain of wild type human FVII only in the presence of at least 0.05 mM of a divalent cation to an immunoaffinity purification column,
(b) applying the sample to the column in the presence of at least 0.05 mM of a divalent cation,
(c) eluting the FVII polypeptides comprising an intact GLA domain from the column by removal of the divalent cation from the column,
wherein the divalent cation is Ca²⁺ present in the range from about 0.05 mM to about 50 mM, such as from about 0.1 mM to about 30 mM, such as from about 1 mM to about 20 mM, such as from about 5 mM to about 10 mM.

The term “EGF-like domain 1” as used herein means the amino acid sequence 46-82 of SEQ ID NO:1. The term “EGF-like domain 2” as used herein means the amino acid sequence 87-128 of SEQ ID NO:1.

Proper folding and activity of FVII is dependent on the presence of calcium, and it has now been found that certain epitopes on FVII are only exposed in the presence of divalent cations such as calcium ions. The presence of metal ions is essential for the formation of and exposure of epitopes in the GLA domain recognized by the antibodies according to the invention. In one embodiment divalent cation is a metal ion. In one embodiment the divalent cation is selected from the list consisting of Zn²⁺, Ca²⁺, Mg²⁺, Cu²⁺, Mn²⁺, Co²⁺, Fe²⁺, Sm²⁺, Ni²⁺, Cd²⁺, Hg²⁺, Sm²⁺, and Uo²⁺. In one embodiment the divalent cation is calcium. In one embodiment the divalent cation is Zn²⁺. In one embodiment the divalent cation is Mg²⁺.

The correct amount of divalent cation can easily be determined by the skilled person, however, normally the cation concentration should at least be 0.05 mM, such as at least 0.1 mM, such as at least 1 mM.

In a particular embodiment the divalent cation is present in an amount above 0.05 mM, such as above 0.1 mM, such as above 0.6 mM, such as above 1 mM, such as above 5 mM.

In a particular embodiment the divalent cation is present in an amount in the range from about 0.05 mM to about 50 mM, such as from about 0.1 mM to about 30 mM, such as from about 0.6 mM to about 30 mM, such as from about 1 mM to about 20 mM, such as from about 5 mM to about 10 mM.

In a particular embodiment Ca²⁺ is present in an amount in the range from about 0.05 mM to about 50 mM, such as from about 0.1 mM to about 30 mM, such as from about 0.6 mM to about 30 mM, such as from about 1 mM to about 20 mM, such as from about 5 mM to about 10 mM.

### Antibodies

The present invention provides novel antibodies and fragments or derivatives thereof that bind the antigen with high affinity to epitopes present in domains irrespective of proper folding and to antibodies the bind to epitopes exposed in the antigen in a correctly processed antigen/polypeptide. These latter antibodies recognize epitopes exposed in the presence of calcium.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention because they are the most common antibodies in the physiological situation and because they are most easily made in a laboratory setting. Preferably the antibody of this invention is a monoclonal antibody.

The antibodies of this invention may be produced by a variety of techniques known in the art. Typically, they are produced by immunization of a non-human animal, preferably a mouse, with an immunogen comprising the FVII polypeptide.

Alternatively a specific antibody may be expressed as recombinant proteins. The specific antibodies of the present invention are meant as examples of suitable antibodies and can be produced from the specific sequences of the variable regions, particularly the hypervariable regions known as Complementary Determining Regions (CDR). A skilled person will from the sequence of the CDR-regions be able to recombinantly express a complete monoclonal antibody as also illustrated in the examples.

The FVII polypeptide may comprise the full length sequence, or a fragment or derivative thereof, typically an immunogenic fragment, i.e., a portion of the FVII polypeptide comprising an exposed epitope.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988)). The immunogen is then suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the pre-sent invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections, are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

For polyclonal antibody preparation, serum is obtained from an immunized non-human animal and the antibodies present therein isolated by well-known techniques. The serum may be affinity purified using any of the immunogens set forth above linked to a solid support so as to obtain antibodies that react with the FVII polypeptide, particularly with Factor VIIa.

In an alternate embodiment, lymphocytes from an un-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. The hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in (Goding, “Monoclonal Antibodies: Principles and Practice,” pp. 59-103 (Academic Press, 1986)).

The cells are allowed to grow in the selection media for sufficient time for colony formation and antibody production. This is usually between 7 and 14 days. The hybridoma colonies are then assayed for the production of antibodies that specifically bind to the FVII polypeptide. The assay is typically a calorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be re-cloned and grown to ensure that only a single cell has given rise to the colony producing the desired antibody. Positive wells with a single apparent colony are typically re-cloned and re-assayed to insure only one monoclonal antibody is being detected and produced.

Antibodies may also be produced by selection of combinatorial libraries of immunoglobulins, as disclosed for instance in Ward et al (Nature 341 (1989) 544).

### Recombinant Production

Antibodies can also be prepared by recombinant expression in single cell organisms, such as yeast; or in bacterial cell cultures (such as in E. coli); or in eukaryotic cell culture (e.g., in a culture of a mammalian cells) using standard techniques.

Thus, according to an alternate embodiment, the DNA encoding heavy and light chains of an anti-FVII antibody is isolated from the hybridoma of this invention and placed in an appropriate expression vector for transfection into an appropriate host. The host is then used for the recombinant production of the antibody, or variants thereof, such as a humanized version of that monoclonal antibody, active fragments of the antibody, or chimeric antibodies comprising the antigen recognition portion of the antibody.

DNA encoding the monoclonal antibodies of the invention is readily isolated and sequenced using conventional procedures (e.g., by using oligonucleotide probes that are capable of binding specifically to genes encoding the heavy and light chains of murine or human antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding fragments of the antibody is well known in the art (see, for example, Skerra et al., Curr. Opinion in Immunol., 5, pp. 256 (1993); and Pluckthun, Immunol. Revs. 130, pp. 151 (1992).

Additionally, recombinant production of antibodies from known variable heavy (VH) and variable light (VL) chains, and human constant regions has been described by, for example, Ruker et al. (Annals of the New York Academy of Sciences. 1991; 646:212-219), who reports the expression of a human monoclonal anti-HIV-1 antibody in CHO cells; Bianchi et al. (Biotechnology and Bioengineering. 2003; 84:439-444), who describes high-level expression of full-length antibodies using trans-complementing expression vectors, No Soo Kim et al. (Biotechnol. Prog. 2001; 17:69-75), who describes key determinants in the occurrence of clonal variation in humanized antibody expression of CHO cells during dihydrofolate reductase mediated gene amplification; King et al. (Biochemical Journal. 1992; 281:317-323), who reports expression, purification and characterization of a mouse-human chimeric antibody and chimeric Fab′ fragment; WO 2003064606 which describes isolated human monoclonal antibodies comprising a human heavy and a human light chain variable regions, both comprising FR1, CDR1, FR2, CDR2, FR3, CDR3 and FR4 sequences; and WO 2003040170 which describes chimeric or human monoclonal antibodies and antigen-binding portions that specifically binds to and activates human CD40.

The entire cDNA sequences encoding the constant regions of human IgG can be found in the following GenBank entries, each of which are incorporated by reference in its entirety, accessed on Jan. 6, 2005:

Human IgG1 constant heavy chain region: GenBank accession #: J00228
Human IgG2 constant heavy chain region: GenBank accession #: J00230
Human IgG3 constant heavy chain region: GenBank accession #: X04646
Human IgG4 constant heavy chain region: GenBank accession #: K01316
Human kappa light chain constant region: GenBank accession #: J00241.

As discussed above antibodies suitable for use in certain methods according to the invention should be used in “matched pairs” meaning that the two antibodies recognize and bind to different epitopes on the FVII polypeptide. The determination of whether specific antibodies bind to different epitopes can be readily determined using any one of a variety of immunological screening assays in which antibody competition can be assessed. All such assays are routine in the art (see, e.g., U.S. Pat. No. 5,660,827, issued Aug. 26, 1997).

According to the techniques described above several specific monoclonal antibodies have been isolated and tested (for details on the expression of these antibodies see the examples and sequence listings) and three monoclonal antibodies, FVII-3F11A3, FVII-3F3A4, and FVII-3F20 μl have been shown to have very high affinity towards the target domain which is the GLA domain of the FVII polypeptide. These antibodies recognize epitopes which are present in the polypeptide dependently of calcium ions and are referred to as calcium dependent binding.

In one embodiment of the invention the method for determination of amounts of FVII polypeptides with an intact GLA domain is performed using any of FVII-3F3A4, FVII-3F11A3, and FVII-3F20 μl as monoclonal antibodies specific for an epitope present on the GLA domain.

The amino acid sequences of the variable light (VL) chain of the antibody FVII-3F3A4 is given in the SEQ ID NO: 3. The amino acid sequences of the variable heavy (HL) chain of the antibody FVII-3F3A4 is given in the SEQ ID NO: 5.

The amino acid sequences of the variable light (VL) chain of the antibody FVII-3F20 μl is given in the SEQ ID NO: 7. The amino acid sequences of the variable heavy (HL) chain of the antibody FVII-3F20 μl is given in the SEQ ID NO: 9.

The amino acid sequences of the variable light (VL) chain of the antibody FVII-3F11A3 is given in the SEQ ID NO: 11. The amino acid sequences of the variable heavy (HL) chain of the antibody FVII-3F11A3 is given in the SEQ ID NO: 13.

The method of the invention has been found to be very convenient for obtaining a fast and reliable measure of the quality of the product, the FVII polypeptide, during culture of a host cell producing the polypeptide.

By applying the method of the invention it is possible to determine the amount of functional FVII polypeptide, which in the present context means a correctly processed FVII polypeptide comprising an intact gamma carboxylated GLA domain, as well as the total amount of the FVII polypeptide present in the culture liquid.

In one embodiment the present invention therefore relates to a method for determining the ratio of correctly processed FVII polypeptide to total amount of the FVII polypeptide in a sample comprising the steps of:

a) determining the amount of the FVII polypeptide comprising a gamma-carboxylated GLA domain having a disulphide bond between cys17 and cys22; and
b) determining the total amount of the FVII polypeptide present in the sample.

The ratio of correctly processed polypeptide to total FVII polypeptide in a sample can thus be calculated, and the ratio is in the present invention termed the calcium dependent index (CDI).

Correct processing of polypeptides comprising a gamma-carboxylated domain, GLA domain, has been shown to be dependent on calcium, possibly for proper folding. This conformational change induced in the presence of calcium might expose epitopes in the folded polypeptide which are not present in propeptide forms, GLA domain-less forms or non-gamma-carboxylated forms of the FVII polypeptide.

In one embodiment determination of the different forms of the FVII polypeptide is done by binding of specific antibodies to different domains in the polypeptide and detecting the amount of the detecting antibodies.

Different detection systems for detecting binding of antibodies to a target antigen is well known in the art and includes e.g. conjugated enzymes (ELISA) and fluorescent linked antibodies.

In a further embodiment the present invention therefore relates to a method for determining the ratio of a correctly processed FVII polypeptide total amount of the FVII polypeptide in a sample, wherein the amount of the FVII polypeptide comprising a correctly processed GLA-domain, and the total amount of the FVII polypeptide present in the sample are determined by detecting binding of a specific detecting antibody directed against an epitope exposed in the presence of Ca²⁺ on the correctly processed GLA-domain in the FVII polypeptide and by detecting binding of another specific detecting antibody directed against any other epitope in a different domain in the FVII polypeptide respectively.

In case a “sandwich” technique is employed in which a catching antibody or antibodies immobilized on a solid support is applied together with a detecting antibody or antibodies, several possible combinations of antibodies can be envisaged.

In one embodiment the catching antibody could be any antibody having a high affinity towards the antigen/polypeptide and which antibody will bind all or most of the forms of the FVII polypeptide. Such an antibody could e.g. bind to epitopes in the EGF-like domain of the FVII polypeptide. The detecting antibodies should then be able to discriminate between the different forms present and one of the detecting antibodies should be specific for an epitope exposed on a GLA domain in the presence of Ca²⁺ and another detecting antibody should be specific for an epitope on a domain which is not a GLA domain and which epitope on the non-GLA domain is different from the epitope recognized by the catching antibody.

The detection of binding of the detecting antibodies, which measures correctly processed or total antigen, are conveniently performed on two separate samples as will be the case when a sandwich ELISA techniques is used, however, performing the detection of both detecting antibodies on the same sample could be envisaged in the case where a fluorescent molecule on the detecting antibodies are measured directly. It would then be necessary to use two different excitation wavelengths.

When on the other hand two different catching antibodies are applied in the method two separate samples are always employed. In this case the two catching antibodies are one catching antibody specific for an epitope exposed on a GLA-domain in the presence of Ca²⁺ and another catching antibody specific for a first epitope in a domain which is not a GLA-domain, and the detecting antibody is an antibody specific for a second epitope in a domain which is not a GLA-domain, wherein the second epitope is different from the first epitope.

As an example one embodiment could be F1 and F9 as catching antibodies and F7 as detecting antibody.

As described above one way of detecting binding of the detecting antibody is by ELISA in which an enzyme conjugated antibody is employed and the amount of antibody bound is determined by a calorimetric assay. Particularly the ELISA is a sandwich ELISA. However, other means for detecting antibody binding can be employed as well.

One well known technique for monitoring biomolecular interactions is by surface plasmon resonance (SPR). Surface plasmon resonance is a phenomenon which occurs when light is reflected off thin metal films. A fraction of the light energy incident at a sharply defined angle can interact with the delocalised electrons in the metal film (plasmon) thus reducing the reflected light intensity. The precise angle of incidence at which this occurs is determined by a number of factors, but in the Pharmacia BIAcore devices the principal determinant becomes the refractive index close to the backside of the metal film, to which target molecules are immobilised and addressed by ligands in a mobile phase running along a flow cell. If binding occurs to the immobilised target the local refractive index changes, leading to a change in SPR angle, which can be monitored in real-time by detecting changes in the intensity of the reflected light, producing a sensor-gram. The rates of change of the SPR signal can be analysed to yield apparent rate constants for the association and dissociation phases of the reaction. The ratio of these values gives the apparent equilibrium constant (affinity). The size of the change in SPR signal is directly proportional to the mass being immobilised and can thus be interpreted crudely in terms of the stoichiometry of the interaction. Signals are easily obtained from sub-microgram quantities of material. Since the SPR signal depends only on binding to the immobilised template, it is also possible to study binding events from molecules in extracts, i.e. it is not necessary to have highly purified components.

Biomolecular interactions occurring at the sensor surface change the solute concentration and thus the refractive index within the evanescent wave penetration range. The angle of incidence required to create the SPR phenomenon (the SPR angle) is therefore altered and it is this change which is measured as the response signal. SPR thus provides a mass detector which is essentially independent of the nature of the interactants. The technique requires no labeling.

Other possible means suitable for detecting interactions between antibodies and antigen is by pieso electric biosensors in which the parameter which is measure is resistance, current or voltage. The target, the FVII polypeptide, is immobilized on a cantilever with a built in pieso resistor, and binding of the detecting antibody will induce bending which strains the piezo-resistor and thereby changes the resistor value. Other possible means of detection can easily be envisaged by the skilled person, e.g. SAW (surface acoustic waves)-biosensors etc.

In one embodiment therefore the detection of binding of the detecting antibody/antibodies is performed by ELISA, surface plasmon resonance, pieso electric biosensors or SAW-biosensors.

The CDI of the invention can be determined for any protein having a GLA domain the correct processing of which correlates to the activity of the protein and wherein the proper folding is affected by the presence of calcium. Such proteins include Factor VII, VIIa, IX, IXa, X, Xa, protein C, protein S, protein Z, osteocalcin, matrix GLA-protein, proline-rich Gla proteins 1 and 2.

The method of calculating the CDI has the advantage that undesirable forms of the polypeptide, e.g. Factor VIIa, such as GLA domainless FVII, pro-FVII, non-gamma-carboxylated-FVII and other forms of FVII with degraded GLA domain, which would be detected in a normal sandwich ELISA, can be discriminated and thus a quality indicator of the culture can be obtained.

It has surprisingly been found that the CDI index during the production of FVII varies to a high extent during the culture period and the knowledge of the CDI is therefore of great importance in order to obtain the best product yield.

A second aspect of the invention therefore relates to a use of the method of the invention for optimizing the yield of the functional FVII polypeptide during production.

### EXAMPLES

### Example 1

### Recombinant Production of Antibodies

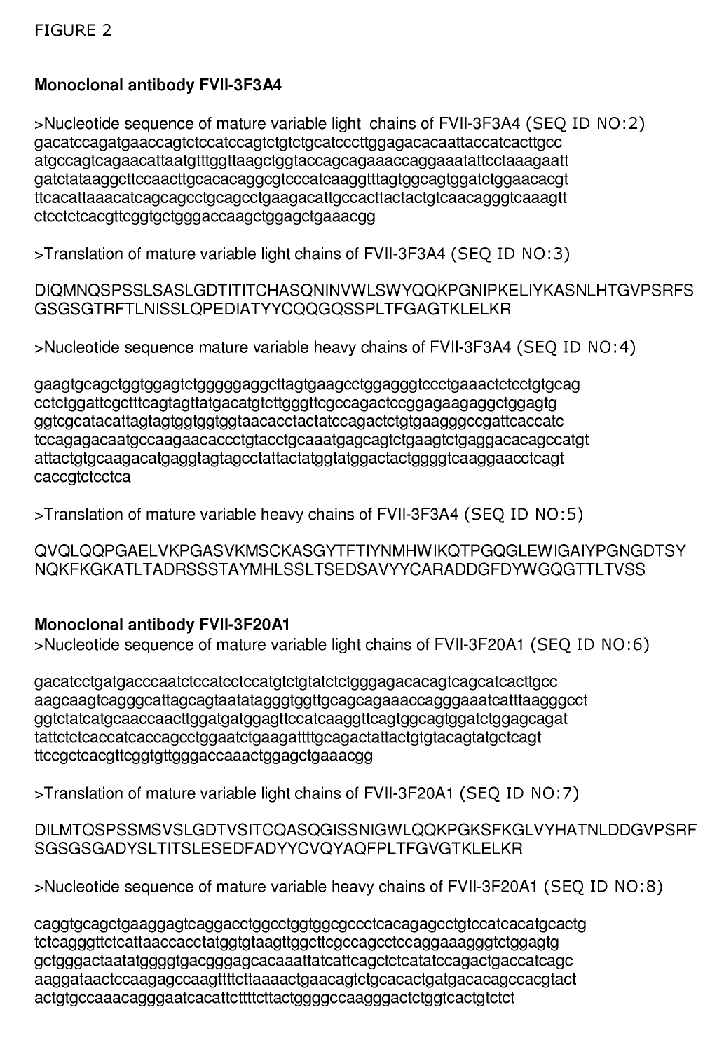

In an exemplary embodiment, to produce recombinant mAb from VH and VL sequences of FVII antibodies, the following protocol can be applied. Steps 1-3 describe retrieval of the VH and VL regions from a hybridoma or other cell producing monoclonal FVII antibody. Alternatively, the cDNA encoding the FVII antibody VH and VL sequences to be used in step 4 can be prepared from the sequence information provided in FIG. 2, using well-established techniques for synthesizing cDNA fragments. The VH and VL fragments of the desired antibody, or mutants or derivatives thereof, may also be cloned into any one of a number of expression vectors described in the scientific literature or commercially available expression vectors, containing a constant region of the desired Ig subclass, in order to express a full-length antibody. Additionally, VH and VL fragments of the desired antibody, or mutants or derivatives thereof can be cloned into vectors encoding truncated constant regions in order to express antibody fragments (e.g., Fab fragments). One example of a commercially available vector is pASK84, available from the ATCC (American Type Culture Collection, catalog number 87094).

(1) Isolation of Total RNA from Hybridoma Cells:

4×10⁶ hybridoma cells secreting antibodies against FVII are used for isolation of total RNA using RNeasy Mini Kit from Qiagen, according to manufacturers instructions, and briefly outlined here: The cells are pelleted by centrifugation for 5 min at 1000 rpm and disrupted by addition of 350 μl RLT buffer containing 10 μl/ml β-mercaptoethanol. The lysate is transferred onto a QIAshredder column from Qiagen and centrifuged for 2 min at maximum speed. The flow-through is mixed with an equal volume of 70% ethanol. Up to 700 μl sample is applied per RNeasy spin column (Qiagen) and centrifuged at 14000 rpm, and the flow-through discarded. 700 μl RW1 buffer is applied per column which is centrifuged at 14000 rpm for 15 s to wash the column. The column is washed twice with 500 μl RPE buffer and centrifuged for 14000 rpm for 15 s. To dry the column it is centrifuged for additionally 2 min at 14000 rpm. The column is transferred to a new collection tube and the RNA is eluted with 50 μl of nuclease-free water and centrifuged for 1 min at 14000 rpm. The RNA concentration is measured by absorbance at OD=260 nm. The RNA is stored at −80° C. until needed.

(2) cDNA Synthesis:

1 μg RNA is used for first-strand cDNA synthesis using SMART RACE cDNA Amplification Kit from Clontech. For preparation of 5′-RACE-Ready cDNA, a reaction mixture is prepared containing RNA isolated as described above, the reverse-primer 5′-CDS primer back, and SMART II A oligo, and this mixture is incubated at 72° C. for about 2 min., and subsequently cooled on ice for about 2 min. before adding 1× First-Strand buffer, DTT (20 mM), dNTP (10 mM) and PowerScript Reverse Transcriptase. The reaction mixture is incubated at 42° C. for 1.5 hour and Tricine-EDTA buffer is added and incubated at 72° C. for 7 min. At this point samples can be stored at −20° C.

### (3) PCR Amplification and Cloning of Human Variable Light (VL) and Human Variable Heavy (VH) Chains:

A PCR (Polymerase Chain Reaction) reaction mixture containing 1× Advantage HF 2 PCR buffer, dNTP (10 mM) and 1× Advantage HF 2 polymerase mix is established for separate amplification of variable regions of both VL and VH from cDNA made as above.

For amplification of VL the following primers are used:

UPM (Universal Primer Mix):

(SEQ ID NO:14)

5′-CTAATACGACTCACTATAGGGCAAGCAGTGGTATCAACGCAGA
GT-3′

(SEQ ID NO:15)

5′-CTAATACGACTCACTATAGGG-3′
VK RACE2:

(SEQ ID NO:16)

5′-GCAGGCACACAACAGAGGCAGTTCCAGATTTC-3′

For amplification of VH the following primers are used:

UPM (Universal Primer Mix):

(SEQ ID NO:17)

5′-CTAATACGACTCACTATAGGGCAAGCAGTGGTATCAACGCAGA
GT-3′

(SEQ ID NO:18)

5′-CTAATACGACTCACTATAGGG-3′
AB90RACE:

(SEQ ID NO:19)

5′-GTGCCAGGGGGAAGACCGATGGG-3′

Three rounds of PCR are conducted. Round 1: PCR is run for 5 cycles at 94° C. for 5 s and 72° C. for 3 min. Round 2: PCR is run for 5 cycles at 94° C. for 5 s, 70° C. for 10 s, and 72° C. for 1 min. Round 3: PCR is run for 28 cycles at 94° C. for 5 s, 68° C. for 10 s, and 72° C. for 1 min.

The PCR products are analyzed by electrophoresis on a 1% agarose gel and the DNA purified from the gel using QIAEX11 agarose gel extraction kit from Qiagen. The purified PCR products are introduced into PCR4-TOPO vector using TOPO TA Cloning kit from Invitrogen and used for transformation of TOP10 competent cells.

A suitable amount of colonies are analyzed by colony PCR using Taq polymerase, 1× Taq polymerase buffer, dNTP (10 mM) and the following primers and PCR program:

(SEQ ID NO:20)

| | |
|---|---|
| M13forward primer: | 5′-GTAAAACGACGGCCAG-3′ |

(SEQ ID NO:21)

| | |
|---|---|
| M13reverse primer: | 5′-CAGGAAACAGCTATGAC-3′ |

### PCR Program:

25 cycles are run at 94° C. for 30 s, 550C for 30 s, and 72° C. for 1 min. Plasmid DNA from clones comprising VL and VH inserts, respectively, is extracted and sequenced using primer M13forward and M13reverse listed above. In the case of a FVII mAb, the sequences encoding the heavy and light chain variable regions are shown in FIG. 2.

(4) Subcloning of Antibody Genes into Mammalian Expression Vectors

Based on the sequence data for cDNAs encoding the heavy and light chain variable regions of the mAb, primers are designed for the amplification of the variable light (VL) and variable heavy (VH) chain genes, respectively. The variable regions are formatted by PCR to include a Kozak sequence, leader sequence and unique restriction enzyme sites. For the VL, this is achieved by designing 5′ PCR primers to introduce a HindIII site, the Kozak sequence and to be homologous to the 5′ end of the leader sequence of the variable light chain region. The 3′ primer is homologous to the 3′ end of the variable region and introduced a BsiWI site at the 3′ boundary of the variable region. The VH region is generated in a similar fashion except that a NotI and a NheI site are introduced in the 5′ and 3′ end instead of HindIII and BsiWI, respectively.

The amplified gene products are each cloned into a eukaryotic expression vector containing the light and heavy chain constant regions, using standard techniques. The VL DNA fragments is digested with HindIII and BsiWI and ligated into a eukaryotic expression vector containing the beta-lactamase gene encoding resistance to ampicillin and an E. coli replication origin (pUC); the resulting plasmid is designated VLCL. The VH DNA fragments, is digested with NotI and NheI and introduced into the VLCL vector resulting from the introduction of VL fragment as described above. The resulting plasmid contains functional expression cassettes encoding both the heavy and light chains of the antibody on the same plasmid. The ligated plasmid is used to transform E. coli. Plasmid DNA is prepared from these ampicillin resistant bacterial populations and used for transfection into Chinese hamster Ovary cells, or other mammalian cell lines. Transfection and cell culture is done by standard methods, as described for example in “Molecular Cloning”, Sambrook et al. The result is transfected cell lines that stably express and secrete the antibody molecule of interest, such as the FVIImAb or a mAb comprising the VH and VL regions of FVII Ab. Variants of the antibody can easily be generated. For example, an antibody with the exact same specificity as e.g. FVII-3F11A3, FVII-3F3A4, and FVII-3F20A1 but of a different isotype than IgG4 can be obtained by sub-cloning the cDNA encoding VL and VH of the Ab of interest into plasmids containing cDNA encoding the kappa light chain constant regions and the IgG1 or IgG2 or IgG3 constant regions. Thus, an antibody as generated can possess any isotype and the antibody can then be isotype switched using conventional techniques in the art. Such techniques include the use of direct recombinant techniques (see, e.g., U.S. Pat. No. 4,816,397), cell-cell fusion techniques (see e.g., U.S. Pat. No. 5,916,771), and other suitable techniques known in the art. Accordingly, the effector function of antibodies provided by the invention may be “changed” with respect to the iso-type of a parent antibody by isotype switching to, e.g., an IgG1, IgG2, IgG3, IgG4, IgD, IgA, IgE, or IgM antibody for various uses, including therapeutic ones.

### Example 2

### Determination of Total Factor VIIa Concentration in a Sample by Sandwich ELISA

The determination of the total Factor VII concentration in a sample can be determined by a sandwich ELISA using any two monoclonal antibodies against two different epitopes, such as anti-EGF domain antibodies:

A 96 well microplate (C96 maxisorp Nunc-Immuno plate from Nalgene Nunc International) was coated overnight at 4° C. with 1 μg of the monoclonal F9 anti-FVII (primary) in 100 μL of coating buffer (0.1 M NaHCO₃, pH 9.8) per well. After incubation, the plate was washed 4 times using 350 μL of washing buffer (20 mM Hepes, 100 mM NaCl, 10 mM CaCl₂, 0.02% Tween 80, pH 7.4). After washing, the wells were blocked for 2.5 hours, using 350 μL of blocking buffer (20 mM Hepes, pH 7.4, 0.1 M NaCl, 10 mM CaCl₂, 1% BSA, 0.02% Tween 80). The blocking was performed using a shaker table at room temperature.

100 μL, 1 μg/ml of monoclonal F7 anti-FVII (secondary) conjugated to peroxidase was added to each well.

Samples were diluted to approximately 20 ng FVII/ml in dilution buffer (20 mM Hepes, pH 7.4, 0.1 M NaCl, 10 mM CaCl₂, 2 mg/ml BSA, 0.02% Tween 80) and 20 μL was loaded into the respective wells. A standard series of doubles at 0, 5, 10, 20, 30 and 50 ng/ml and a number of controls were also added. After loading, the plate was incubated for two hours at room temperature on a shaker table.

The plate layout was laid as follows:

TABLE 1
ELISA plate layout.

| | 1 | 2 | 3 | 4 | 5 | 6 | 7 | 8 | 9 | 10 | 11 | 12 |
|---|---|---|---|---|---|---|---|---|---|---|---|---|
| A | Blind | Std 30 | C2 | U3 | U7 | U11 | U15 | U19 | U23 | U27 | C3 | Std 20 |
| B | Blind | Std 30 | C2 | U3 | U7 | U11 | U15 | U19 | U23 | U27 | C3 | Std 20 |
| C | Std 5 | Std 50 | C3 | U4 | U8 | U12 | U16 | U20 | U24 | U28 | Blind | Std 30 |
| D | Std 5 | Std 50 | C3 | U4 | U8 | U12 | U16 | U20 | U24 | U28 | Blind | Std 30 |
| E | Std 10 | | U1 | U5 | U9 | U13 | U17 | U21 | U25 | C1 | Std 5 | Std 50 |
| F | Std 10 | | U1 | U5 | U9 | U13 | U17 | U21 | U25 | C1 | Std 5 | Std 50 |
| G | Std 20 | C1 | U2 | U6 | U10 | U14 | U18 | U22 | U26 | C2 | Std 10 | |

Std xx = standard xx ng/ml,
C = control,
U = sample

After incubation, the plates were washed five times using 350 μL of washing buffer (20 mM Hepes, 100 mM NaCl, 10 mM CaCl₂, 0.02% Tween 80, pH 7.4). 100 μL of substrate (100 mM OPD (orto-phenylendiamine), 1 mM H₂O₂ in 50 mM NaAc, 1 mM CaCl2, pH 5.2) was added to each well and the plate were left to develop on a shaker table at room temperature and stopped by adding 1.25 M H₂SO₄ once the high control had reached an OD of approximately 1.2. The plate was read in an ELISA plate reader using a 492 nm filter.

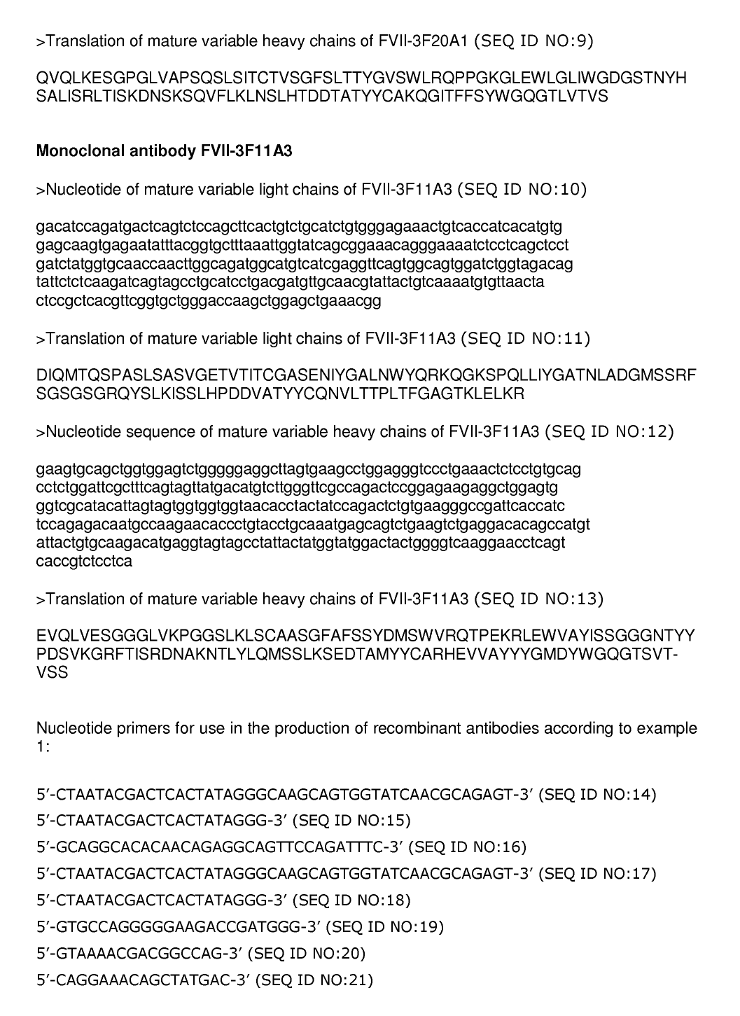

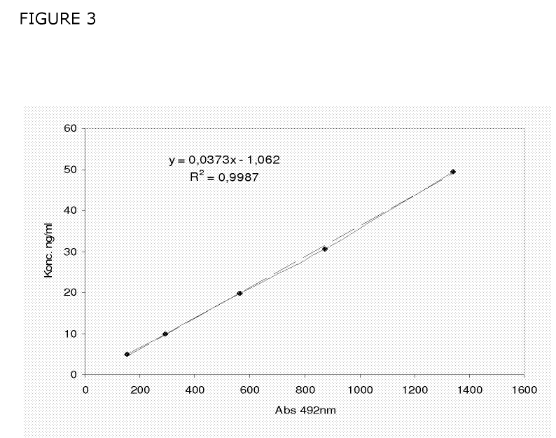

The standard curve shown in FIG. 3 was used to determine the FVII concentrations in the individual wells.

### Example 3

### Control of Production Parameters for the Production of Active Factor VIIa

A batch or continuous cell culture can be monitored for it's total production of FVII using the standard FVII ELISA described in Example 2. The specific content and hence production of FVII which includes an intact GLA domain can be determined using the following sandwich ELISA assay, based on an antibody against the GLA domain and an antibody against another epitope of FVII.

A 96 well microplate (C96 maxisorp Nunc-Immuno plate from Nalgene Nunc International) was coated overnight at 4° C. with 5 μg of the monoclonal F1 anti-FVII (primary) in 100 μL of coating buffer (50 μg/ml F1A2 i 20 mM Hepes, 100 mM NaCl, 10 mM CaCl₂, pH 7.4) per well. After incubation, the plate was washed 4 times using 350 μL of washing buffer (20 mM Hepes, pH 7.4, 0.1 M NaCl, 10 mM CaCl₂, 0.2% Tween 80). After washing, the wells were blocked for 2.5 hours, using 350 μL of blocking buffer (20 mM Hepes, pH 7.4, 0.1 M NaCl, 10 mM CaCl₂, 1% BSA, 0.02% Tween 80). The blocking took 2.5 hours and was performed using a shaker table at room temperature.

100 μL, 1 μg/ml of monoclonal F7 anti-FVII (secondary) conjugated to peroxidase was added to each well.

Samples were diluted to approximately 75 ng FVII/ml in dilution buffer (20 mM Hepes, pH 7.4, 0.1 M NaCl, 10 mM CaCl₂, 2 mg/ml BSA, 0.02% Tween 80) and 20 μL was loaded into the respective wells. A standard series of doubles at 0, 20, 30, 50, 80, 100 and 130 ng/ml and a number of controls were also added. After loading, the plate was incubated for two hours at room temperature on a shaker table.

The plate layout was laid as follows:

TABLE 2
ELISA plate layout.

| | 1 | 2 | 3 | 4 | 5 | 6 | 7 | 8 | 9 | 10 | 11 | 12 |
|---|---|---|---|---|---|---|---|---|---|---|---|---|
| A | Blind | Std 80 | C2 | U3 | U7 | U11 | U15 | U19 | U23 | U27 | C3 | Std 50 |
| B | Blind | Std 80 | C2 | U3 | U7 | U11 | U15 | U19 | U23 | U27 | C3 | Std 50 |
| C | Std 20 | Std 100 | C3 | U4 | U8 | U12 | U16 | U20 | U24 | U28 | Blind | Std 80 |
| D | Std 20 | Std 100 | C3 | U4 | U8 | U12 | U16 | U20 | U24 | U28 | Blind | Std 80 |
| E | Std 30 | Std 130 | U1 | U5 | U9 | U13 | U17 | U21 | U25 | C1 | Std 20 | Std 100 |
| F | Std 30 | Std 130 | U1 | U5 | U9 | U13 | U17 | U21 | U25 | C1 | Std 20 | Std 100 |
| G | Std 50 | C1 | U2 | U6 | U10 | U14 | U18 | U22 | U26 | C2 | Std 30 | Std 130 |
| H | Std 50 | C1 | U2 | U6 | U10 | U14 | U18 | U22 | U26 | C2 | Std 30 | Std 130 |

Std xx = standard xx ng/ml,
C = control,
U = sample

After incubation, the plates were washed five times using 350 μL of washing buffer (20 mM Hepes, pH 7.4, 0.1 M NaCl, 10 mM CaCl2, 0.02% Tween 80). 100 μL of substrate (100 mM OPD (orto-phenylendiamine), 1 mM H₂O₂ in 50 mM NaAc, 1 mM CaCl2, pH 5.2) was added to each well and the plate were left to develop on a shaker table at room temperature and stopped by adding 1.25 M H₂SO₄ once the high control had reached an OD of approximately 1.2. The plate was read in an ELISA plate reader using a 492 nm filter.

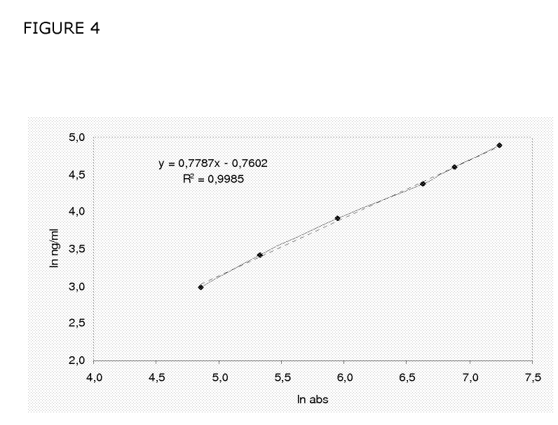

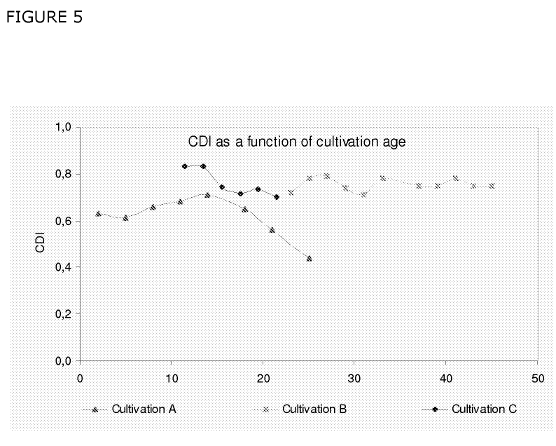

The standard curve shown in FIG. 4 was used to determine the FVII concentrations in the individual wells.

### Example 4

### The Use of CDI to Monitor FVII Cultivations

The Calcium Dependent Index (CDI) is defined as the ratio between FVII responding in the ELISA using at least one GLA domain specific antibody (e.g. FVII-3F11A3, FVII-3F3A4, or FVII-3F20 μl) and the total FVII responding in an ELISA using any other antibodies such as anti EGF (e.g. F7 or F9).

### Example 5

### Use of Antibody for Immunoaffinity Purification

Performing immunoaffinity purification in the presence of 20 mM Ca²⁺

A 1000 ml portion of BHK-21 culture supernatant, stabilized by the addition of calcium to a concentration of 10 mM Ca²⁺ and by the addition of tris buffer to a concentration of 10 mM and subsequent adjustment with HCl to pH 8 is filtered through a 0.45 micron dead-end filter. The stabilized culture supernatant is loaded onto a column (1.6 cm inner diameter×10 cm length=20 ml CV) packed with a Ca²⁺-dependent monoclonal antibody FVII-3F3A4, immobilized onto Pharmacia Sepharose 4B. Prior to loading, the column is equilibrated with 5 CV's of 10 mM CaCl2, 10 mM tris, pH 8. After loading, the column is washed with 2 M NaCl, 10 mM CaCl2, 10 mM tris, pH 8 for 10 CV's. The bound FVII is eluted with 10 CV's of 30 mM EDTA, 50 mM tris, pH 8. A flowrate of 12 CV/h and a temperature of 5 degrees Celsius is used throughout the purification. The eluate is immediately stabilized by the addition of calcium chloride to a final concentration of 50 mM.

1. A monoclonal antibody that binds to an epitope present on an intact gamma-carboxyglutamic acid domain of wild type human FVII, wherein said binding requires the presence of at least 0.05 mM of a divalent cation.

2. The monoclonal antibody according to claim 1, wherein said epitope comprises an amino acid selected from the group consisting of Phe4, Leu5, Gla6, Gla7, Leu8, Pro10, Gly11, Gla14, Arg15, Gla16, Cys17, Gla19, Gla20, Cys22, Gla25, Gla26, Ala27, Gla29, Phe31, Lys32, and Gla35 of SEQ ID NO:1.

3. The monoclonal antibody according to claim 1, wherein said monoclonal antibody competes with an antibody is selected from the group consisting of:
(a) An antibody comprising a light chain variable region comprising the amino acid sequence of SEQ ID NO:3, and a heavy chain variable region comprising the amino acid sequence of SEQ ID NO:5;
(b) An antibody comprising a light chain variable region comprising the amino acid sequence of SEQ ID NO:7, and a heavy chain variable region comprising the amino acid sequence of SEQ ID NO:9; and
(c) An antibody comprising a light chain variable region comprising the amino acid sequence of SEQ ID NO:11, and a heavy chain variable region comprising the amino acid sequence of SEQ ID NO:13.

4. The monoclonal antibody according to claim 3, wherein said monoclonal antibody is selected from the group consisting of:
(a) An antibody comprising a light chain CDR1 variable region comprising an amino acid sequence corresponding to residues 24-34 of the amino acid sequence of SEQ ID NO:3, a light chain CDR2 variable region comprising an amino acid sequence corresponding to residues 50-56 of the amino acid sequence of SEQ ID NO:3, a light chain CDR3 variable region comprising an amino acid sequence corresponding to residues 89-95 of the amino acid sequence of SEQ ID NO:3, and a heavy chain CDR1 variable region comprising an amino acid sequence corresponding to residues 33-35 of the amino acid sequence of SEQ ID NO:5, a heavy chain CDR2 variable region comprising an amino acid sequence corresponding to residues 50-64 of the amino acid sequence of SEQ ID NO:5, a heavy chain CDR3 variable region comprising an amino acid sequence corresponding to residues 99-112 of the amino acid sequence of SEQ ID NO:5;
(b) An antibody comprising a light chain CDR1 variable region comprising an amino acid sequence corresponding to residues 24-34 of the amino acid sequence of SEQ ID NO:7, a light chain CDR2 variable region comprising an amino acid sequence corresponding to residues 50-56 of the amino acid sequence of SEQ ID NO:7, a light chain CDR3 variable region comprising an amino acid sequence corresponding to residues 89-95 of the amino acid sequence of SEQ ID NO:7, and a heavy chain CDR1 variable region comprising an amino acid sequence corresponding to residues 33-35 of the amino acid sequence of SEQ ID NO:9, a heavy chain CDR2 variable region comprising an amino acid sequence corresponding to residues 50-64 of the amino acid sequence of SEQ ID NO:9, a heavy chain CDR3 variable region comprising an amino acid sequence corresponding to residues 99-112 of the amino acid sequence of SEQ ID NO:9; and
(c) An antibody comprising a light chain CDR1 variable region comprising an amino acid sequence corresponding to residues 24-34 of the amino acid sequence of SEQ ID NO:11, a light chain CDR2 variable region comprising an amino acid sequence corresponding to residues 50-56 of the amino acid sequence of SEQ ID NO:11, a light chain CDR3 variable region comprising an amino acid sequence corresponding to residues 89-95 of the amino acid sequence of SEQ ID NO:11, and a heavy chain CDR1 variable region comprising an amino acid sequence corresponding to residues 33-35 of the amino acid sequence of SEQ ID NO:13, a heavy chain CDR2 variable region comprising an amino acid sequence corresponding to residues 50-64 of the amino acid sequence of SEQ ID NO:13, a heavy chain CDR3 variable region comprising an amino acid sequence corresponding to residues 99-112 of the amino acid sequence of SEQ ID NO:13.

5.-8. (canceled)

9. A nucleic acid molecule encoding a monoclonal antibody as defined in claim 1.

10. A vector comprising the nucleic acid molecule as defined in claim 9.

11. A cell comprising the vector as defined in claim 10.

12. A method for determining the amount of FVII polypeptides comprising an intact gamma-carboxyglutamic acid domain in a sample said method comprising the steps of:
(a) bringing the sample in contact with a first monoclonal antibody according to claim 1 in the presence of at least 0.05 mM of a divalent cation;
(b) allowing any of the FVII polypeptides present in the sample to bind to said first monoclonal antibody to form a first antibody complex;
(c) bringing said first antibody complex in contact with a detectable second monoclonal antibody specific for a second epitope present on said FVII polypeptide, said second epitope being different from the epitope of said first monoclonal antibody;
(d) allowing said first antibody complex to bind to said detectable second monoclonal antibody to form a second antibody complex; and
(e) detecting the amount of said second antibody complex by detecting the amount of second monoclonal antibody present in the second antibody complex

13. (canceled)

14. The method according to claim 12, wherein the second epitope is present on the EGF-like domain 1 or EGF-like domain 2 of said FVII polypeptide.

15.-19. (canceled)

20. A method for determining the ratio of FVII polypeptides comprising an intact gamma-carboxyglutamic acid domain to total amount of the FVII polypeptide in a sample comprising the steps of:
(a) determining the amount of the FVII polypeptides comprising an intact gamma-carboxyglutamic acid domain by use of method according to claim 12; and
(b) determining the total amount of FVII polypeptide present in the sample.

21. (canceled)

22. A method for the purification of FVII polypeptides comprising an intact gamma-carboxyglutamic acid domain from a samples said method comprising the steps of:
(a) coupling of an antibody according to 1 to an immunoaffinity purification column;
(b) applying said sample to said column in the presence of at least 0.05 mM of a divalent cation; and
(c) eluting said FVII polypeptides comprising an intact gamma-carboxyglutamic acid domain from the column by removal of the divalent cation from the column.

23. (canceled)
